(19) United States
(12) Patent Application Publication
(10) Pub. No.: US 2013/0115569 A1
(43) Pub. Date: May 9, 2013

(54) Method and Related Devices for Mixing Two Injectable Compositions Prior to Injection

(71) Applicants: Gregory Lambert, Chatenay-Malabry (FR); Jurgen Wollbold, Labruguiere (FR); Christophe Combes, Mazamet (FR)

(72) Inventors: Gregory Lambert, Chatenay-Malabry (FR); Jurgen Wollbold, Labruguiere (FR); Christophe Combes, Mazamet (FR)

(73) Assignee: SOFIC (STÉ FRANÇAISE D'INSTRUMENTS DE CHIRURGIE), Mazamet (FR)

(21) Appl. No.: 13/809,837

(22) PCT Filed: Jul. 12, 2011
(86) PCT No.: PCT/EP2011/061892

§ 371 Date: Jan. 11, 2013

(30) Foreign Application Priority Data
Jul. 12, 2010 (EP) 10305774.1

(51) Int. Cl. A61M 5/315

(52) U.S. Cl. CPC A61M 5/31596
USPC 433/90; 433/215

(57) ABSTRACT

This invention relates to a method for mixing a first injectable liquid pharmaceutical composition and a second injectable composition within an injection device, said injection device including: ·a generally cylindrical shell in which a cartridge is lodged or a generally cylindrical barrel, ·at one first end of the generally cylindrical barrel or the generally cylindrical shell, a plunger, the plunger being mounted on the barrel or shell, ·at a second end opposite the first end of the generally cylindrical barrel or the generally cylindrical shell, is provided a needle assembly support member, on which may be mounted a needle assembly, said needle assembly comprising means to be mounted on the needle support member, and further comprising: -at least one hollow cannula for transportation of liquid from the barrel or cartridge to the body of the patient; -and at least one hub surrounding part of the cannula and attached to cannula; said hub possibly including a hollow part; characterized in that the first injectable composition is within the cylindrical barrel or within the cartridge lodged in the cylindrical shell and the second injectable composition is associated with the needle assembly, and further characterized in that the mixture of said first and said second composition occurs in the hub, in the cartridge, or in the cannula or the barrel. The invention also relates to said needle assembly and to said injection device.

### FIELD OF INVENTION

This invention relates to the field of injectable compositions. More specifically, this invention relates to a method for mixing two injectable compositions prior to injection. This invention also relates to an injection device hereinafter called a syringe, comprising two distinct pharmaceutical compositions and means for mixing both compositions prior to injection. More specifically, this invention relates to an injection device suitable for mixing a first injectable composition with a second injectable composition prior to injection, comprising a needle assembly including the second injectable composition.

In a particular embodiment, this invention relates to a method and a device for neutralizing and/or buffering at least one injectable composition having a non-physiological pH by contacting it, prior to injection, with a second composition designed for neutralizing and/or buffering the pH of the first composition.

The injection device of the invention may be used, in particular but not exclusively, in dentistry applications. The first composition may be, for example, a local anesthetic composition for dentistry purposes and the second composition may be, for example, a buffer suitable for buffering said local anesthetic composition. The invention is especially useful for local anesthesia, but also for loco-regional anesthesia and regional anesthesia as well.

### BACKGROUND OF INVENTION

This issue of mixing two injectable compositions, for various purposes, prior to injection, is well-known in the art.

It is known that the storage abilities of a number of pharmaceutical compositions are directly linked to their acidic or basic pH, and that a buffering process has to be implemented extemporaneously shortly prior to injection.

Particularly, the issue of buffering a composition to be injected, having a non-physiological pH, prior to injection, is a constant issue in the field of dental anesthetics.

For example, US 2009/221,984 describe a method and apparatus for buffering lidocaine (with and without epinephrine) while improving shelf life without the need to refrigerate. Neutralizing the pH of dental anesthetics prior to injection may also improve efficacy of these anesthetics, while lowering the pain of the patient during injection and following minutes.

Nevertheless, the operator/practitioner usually performing the surgical act of injecting these compositions is highly reluctant to any amendment or modification of his/her surgical gesture and to any complicated procedure to be performed extemporaneously. Moreover, the manufacturers of cartridges are also reluctant to any modification of their containers and the content thereof, as a mere modification may lead to a loss in stability or liability of the product. There is thus still a need for a very simple method and device for mixing two compositions prior to injection.

This invention thus addresses the issue of mixing, prior to injection, a composition enclosed in a cartridge or a barrel. More specifically, this invention addresses the issue of buffering, prior to injection, a composition enclosed in a cartridge and having a non-physiological pH, using a regular injection device and, anytime possible, letting the operator keep its usual surgical gesture of injection.

When considering the dental field, the method and device of the invention are especially advantageous in that they are safe and provide an efficient solution for preventing pain occurring when a patient is injected with a dental anesthetic. Especially, when the site of injection is an inflammatory area, where the local pH is lowered than regular body pH due to inflammation, the injection of an acidic composition of anesthetic may be very painful. The present invention brings a solution for pain prevention.

Moreover, some dental anesthetics are activated after injection by elevation of their pH at contact with the tissues and body fluids having a regular pH of about 6.8 to 7.4: when the tissues are in an inflammatory area and have a lower pH, activation of the dental anesthetics cannot correctly be performed in situ and the success rate of the anesthesia is lowered. This invention provides a solution for enhancing the success rate of anesthesia, even on an inflammatory site.

Most importantly, mixing a buffer with a dental anesthetic may also improve duration of action and/or efficacy of the dental anesthetic.

### DEFINITIONS

In the present invention, the following terms have the following meanings:
“injectable composition” means a composition suitable for being introduced in animal, including human, body or its parts, for example but not exclusively by intradermal, hypodermal, intramuscular, intravenous, or epidural administration routes. It is sterile;
“composition” may be a solid (such as for example a powder), a fluid or a liquid such as for example a solution, a suspension, an emulsion);
“activator” means a second composition having the ability to enhance the properties, such as the efficacy or the biodisponibility of a first composition;
“pH-adjuster”: means an injectable composition capable of lowering or enhancing or buffering the pH of another injectable composition by simple contact between both compositions;
“physiological pH” is a pH ranging from 6.5 to 7.8;
A composition is deemed “not having a physiological pH” when the pH of said composition is below 6.5 or over 7.8;
“injection device” relates to any means suitable for injection in animal body or its parts, for example but not exclusively by intradermal, hypodermal, intramuscular, intravenous, or epidural administration routes; according to the invention, an injection device may comprise a regular syringe, a cartridge syringe, a disposable syringe or a reusable syringe;
“regular syringe” relates to a syringe that contains directly the injectable composition in its barrel;
“cartridge syringe” relates to a syringe in which a cartridge may be adapted;
“cartridge” means a cartridge, generally containing a pharmaceutical composition;
“cylindrical shell” relates to the part of a syringe capable of containing a cartridge.
“cylindrical barrel” means the part of a syringe capable of containing directly a pharmaceutical composition;
“cannula” refers to a hollow flexible tube, usually containing a trocar at one end, that is inserted into a bodily cavity, duct, tissue or vessel, to administer a substance such as a pharmaceutical composition;
“needle assembly” includes a cannula and at least one hub surrounding part of the cannula and attached to cannula;
“needle assembly support member” means the part of the device through which the needle assembly may be connected to the cylindrical barrel or to the cartridge;
“hub” refers to a piece capable of connecting a cannula to the needle member support of a device according to the invention, or more generally to a syringe body;
“plunger” is used for piston or for any piece with a motion similar to that of a piston.
“prior to injection” means at most one week, preferably at most 48 hours, more preferably at most 12 hours, even more preferably at most 6 hours before injection;

### SUMMARY

This invention thus relates to a method for mixing a first injectable liquid pharmaceutical composition and a second injectable composition within an injection device, said injection device including:
a generally cylindrical shell in which a cartridge is lodged or a generally cylindrical barrel,
at one first end of the generally cylindrical barrel or the generally cylindrical shell, a plunger, the plunger being mounted on the barrel or shell,
at a second end opposite the first end of the generally cylindrical barrel or the generally cylindrical shell, is provided a needle assembly support member, on which may be mounted a needle assembly, said needle assembly comprising means to be mounted on the needle support member, and further comprising:
   at least one hollow cannula for transportation of liquid from the barrel or cartridge to the body of the patient,
   and at least one hub surrounding part of the cannula and attached to cannula; said hub possibly including a hollow part;
characterized in that the first injectable composition is within the cylindrical barrel or within the cartridge lodged in the cylindrical shell and the second injectable composition is associated with the needle assembly, further characterized in that the injection device includes means for mixing the first and second composition, and the mixture of said first and said second composition occurs in the hub, in the cartridge or the barrel, or in the cannula.

According to one embodiment, the mixture of the first and the second composition is performed prior to injection and occurs in the hub, in the cartridge or barrel, or in the cannula.

According to an embodiment, the first composition is an injectable composition having a non-physiological pH and the second composition is a buffer of the first composition. In this embodiment, the method of the invention is a method for buffering a first composition with a second composition. Advantageously, the second composition has a pK of 6.5 to 7.8, preferably 7 to 7.4. Preferably, the buffer is NaHCO3, a salt of citric acid, a salt of phosphoric acid or a combination thereof, or any equivalent buffer known from one skilled in the art, having the capacity of buffering the first composition to a pH of 6.5 to 7.8, preferably 7 to 7.4, preferably those described in the European and/or US Pharmacopeia.

According to another embodiment, the second composition is an activator of the first composition. In this embodiment, the method of the invention is a method for activating a first composition with a second composition.

According to one embodiment, the second composition is in the form of a solid, preferably a powder or in the form of a liquid, preferably a solution or an emulsion.

In a particular aspect of the method, the first composition is a liquid and the second composition is in the form of a solid, preferably a powder. In this embodiment, advantageously, the second composition is highly soluble in the first composition.

In another aspect of the method, the first composition is a liquid and the second composition is also in the form of a liquid, preferably a solution or an emulsion. Advantageously, the two liquid compositions are miscible. Preferably, the two compositions are polar compositions. In a preferred embodiment, the ratio of the volume of the second composition to the volume of the first composition is less than one, preferably less than 0.1, more preferably less than 0.05, even more preferably less than 0.03. In a preferred embodiment, the ratio of the volume of the second composition to the volume of the first composition ranges from 0.01 to 0.03.

According to a first embodiment, the volume of the first composition ranges from 1.7 to 1.8 ml or is of about 2.2 ml, when in liquid form.

According to another embodiment, the volume of the second composition, preferably a buffer, is of about 50 microliters when in liquid form.

According to the invention, the second composition is associated with the needle assembly; preferably, the term “associated with” means that the second composition is located on or in the cannula, or inside the hub. According to a first embodiment, the second composition is on the cannula. In this embodiment, of course, the second composition is only present on the part of the cannula that is within the hub and/or that will penetrate into the cartridge or the cylindrical barrel. According to a second embodiment, the second composition is in the cannula. According to a third embodiment, the second composition is in the hub. According to a fourth embodiment, the second composition is coated on interior walls of the hub. According to an embodiment, the second composition may be coated onto the interior and/or outer walls of the cannula(s). In this embodiment, the second composition is preferably in a solid form. In this embodiment, of course, the coated outer walls are located within the hub.

According to an embodiment, the second composition is enclosed in a container surrounding the cannula, the container being preferably located in the part of the cannula which is inside the cartridge or the cylindrical barrel containing the first composition, and the container being capable to release the second composition in the cartridge or in the cylindrical barrel.

According to an embodiment, the first composition is an anesthetic composition, with or without a vasoconstrictor. The anesthetic may preferably be selected from dental anesthetic, preferably from the group comprising or consisting of lidocaine, mepivacaine, prilocaine and articaine and mixtures thereof. The vasoconstrictor may preferably be selected from the group consisting of epinephrine and levonordefin, and mixtures thereof. Preferably, the first composition is selected from lidocaine with or without epinephrine, mepivacaine with or without levonordefin, prilocaine with or without epinephrine, articaine with or without epinephrine. In an embodiment, the first composition is selected from:
lidocaine HCl injection 2%,
mepivacaine HCl injection USP, Plain, 3%,
Prilocaine HCl injection USP, Plain, 4%,
Articaine HCl With Epinephrine USP, 4% 1:100,000,
Articaine HCl With Epinephrine USP, 4% 1:200,000,
lidocaine With Epinephrine injection USP, 2% 1:50,000 to 1:200,000
Mepivacaine HCl With Levonordefrin Injection USP, 2% 1:20,000
Prilocaine With Epinephrine Injection USP, 4% 1:200,000
Bupivacaine With Epinephrine Injection USP, 0.5% 1:200,000.

This invention also relates to a needle assembly comprising means to be mounted on a needle support member of an injection device, preferably on the injection device of the invention, the needle assembly further comprising:
at least one hollow cannula for transportation of liquid from a barrel or cartridge to the body of the patient,
and at least one hub surrounding part of the cannula and attached to cannula; said hub possibly including a hollow part; and the needle assembly includes an injectable composition, preferably a buffer, more preferably NaHCO3, which is associated with the needle assembly.

In an embodiment, the needle assembly of the invention includes at least two cannulas, for example, one of the cannula for injection to the patient, and a second cannula which may be part of the means of mixing of the injection device. In another embodiment, the needle assembly includes a protective sheath, which is fixed on the hub.

In a preferred embodiment, the needle assembly of the invention may be a needle assembly wherein a cannula is extending from outside the part of the hub directed to the patient to outside the part of the hub directed to the needle support member; part of said cannula being surrounded by a further cannula extending from inside the hub to outside the part of the hub directed to the needle support member;
and wherein the hub is divided into three pieces, a lower hub piece, a upper hub piece and a piston; the lower hub piece being connectable to a cylindrical shell or a cylindrical barrel; the upper hub piece comprising at least one opening; the piston being a piston slidably movable in axial direction towards upper hub piece and fixed onto said upper hub piece;
the upper hub piece, the lower hub piece and cannula being connected by a seal; the upper hub piece, the seal and the piston defining a hollow chamber; the second composition being contained in said hollow chamber.

The piece of the hub called piston in this embodiment may be part of the means for mixing the first and the second composition in the method of the invention wherein the injection device includes this needle assembly.

It is hereby made clear that the method of the invention comprises adapting, whenever necessary, a cartridge in the cylindrical barrel, adapting the needle assembly to the needle assembly support member and implementing the mixing by a shift of the plunger, a displacement of the cartridge or the exercise of a force on the needle assembly.

When the injection device used in the method of the invention includes the particular needle assembly as above described, the mixing is operated by the sliding of piston of the needle assembly towards the support member. In an embodiment, the device further comprises a protective sheath associated or fixed with the piston, and the mixing is operated by sliding towards the cartridge the protective sheath, which results in the sliding of the piston. In this embodiment, it may be considered that the piston is actioned through the protective sheath. Preferably, the sheath is of a length such that the cannula remains protected when the sheath is actioned for the mixing. This embodiment is of particular interest as, the operator, in one familiar gesture, may operate the mixing of the first and the second composition, while keeping the cannula protected.

In an embodiment of the invention, the injection device comprises:
a generally cylindrical shell in which a cartridge is lodged or a generally cylindrical barrel,
at one first end of the generally cylindrical barrel or the generally cylindrical shell, a plunger, the plunger being mounted on the barrel or shell,
at a second end opposite the first end of the generally cylindrical barrel or the generally cylindrical shell, is provided a needle assembly support member;
   and is characterized in that it further comprises a needle assembly of the invention as described above, mounted on the needle assembly support member.

This invention also relates to an injection device, which in a first aspect, may be defined as comprising:
a generally cylindrical shell in which a cartridge is lodged or a generally cylindrical barrel,
at one first end of the generally cylindrical barrel or the generally cylindrical shell, a plunger, the plunger being mounted on the barrel or shell
at a second end opposite the first end of the generally cylindrical barrel or the generally cylindrical shell, is provided a needle assembly support member, on which may be mounted a needle assembly, said needle assembly comprising means to be mounted on the needle support member, and further comprising
   at least one hollow cannula for transportation of liquid from the barrel or cartridge to the body of the patient,
   and at least one hub surrounding part of the cannula and attached to cannula; said hub possibly including a hollow part;
      characterized in that a first injectable composition is within the cylindrical barrel or within the cartridge lodged in the cylindrical shell and a second injectable composition is associated with the needle assembly, and further characterized in that the injection device includes means for mixing the first and the second composition, and the mixture of said first and said second composition occurs in the hub, in the cartridge or the barrel, or in the cannula.

In an embodiment, the needle assembly support member comprises together a thread arranged to cooperate with the needle assembly, more specifically with a corresponding thread on a hub or the needle assembly. When the needle assembly is cooperating with, i.e. reversibly or irreversibly fixed to, the needle assembly support, one end of the cannula is within the cartridge or cylindrical barrel, whereas the other end extends outside the hub for use for injection into the patient's tissue.

In another aspect of the invention, the hub in itself defines a hollow part capable of receiving the second composition, either in a liquid or in a solid form. In another embodiment, the hub is mounted on the needle support member and together they define a hollow chamber capable of receiving the second composition, either in a liquid or in a solid form. In an embodiment, the hub comprises at least two parts, one hub piece being mounted on the needle assembly support member of the injection device, and the other one being attached to the cannula, both parts of the hub being connected one to another and possibly defining a hollow chamber. In an embodiment, the two parts of the hub are adapted to slide relatively to each other.

In an embodiment, the mixing means may include the plunger, as it has the function of engaging the barrel or cartridge and of applying pressure to the first composition to dispense the first composition through the needle assembly. As a matter of example, when the plunger is pushed forwardly, it will apply pressure to the first composition within the cartridge or barrel so as to dispense the composition through the needle assembly and contact the second composition stored in the needle assembly, so that both compositions are mixed. After this first action, a further action on the plunger may dispense the mixture outside the device through the cannula.

In one embodiment, the mixing means may include a cannula, especially when the needle assembly comprises at least two cannulas as mentioned above. In another embodiment, the mixing means may include a piston, parts of the hub of the needle assembly as described.

According to one embodiment, the mixing of the first and the second compositions may be performed through a dynamic motion, such as pushing, pulling, pressing, squeezing or sliding at least one part of the injection device, resulting in putting into contact first and second composition within the injection device.

According to one embodiment, the mixing of the first and the second compositions may be performed through a shift of the plunger, an action leading to a displacement of the cartridge, or an action on the needle assembly.

In an embodiment, the dynamic motion is a translation, in axial direction. In another embodiment, the dynamic motion is a rotation.

In a specific embodiment, the dynamic motion is a sliding of the piston piece of the hub toward the support member of the needle assembly.

According to a further aspect of the invention, the mixing of first and second composition may result from the displacement, preferably from the first displacement, of the first composition. Consequently, any action or means resulting in the first displacement of the first composition to the needle assembly may be considered as a suitable mixing means. According to another aspect of the invention, the mixing of first and second compositions may result from the displacement, preferably from the first displacement, of the second composition. Consequently, any action or means resulting in the first displacement of the second composition to the cartridge or barrel may be considered as a suitable mixing means.

According to an embodiment, the needle assembly of the invention is disposable. According to a preferred embodiment, the needle assembly of the invention is sterile.

According to an embodiment, the injection device of the invention is a disposable syringe, intended for one single use only, preferably packed in a bag. In this embodiment, preferably, the device of the invention may be sterile. In this embodiment, the device of the invention may include a fixed barrel prefilled with the first composition and a needle assembly prefilled with the second composition.

According to another embodiment, the injection device of the invention is reusable and may be referred to as a cartridge syringe: a cartridge syringe facilitates repeated use of the device, specifically repeated loading and discharges of cartridges containing a specific dose of first composition. A cartridge syringe is characterized by the absence of a fixed barrel in which a piston reciprocates. Instead, a cartridge syringe includes a cylindrical shell with an opening designed to receive and secure a prefilled cartridge including first composition, the ends of which cartridge are closed and hermetically sealed. The device is provided with a reciprocatable plunger arranged to engage with one end of the cartridge.

According to an embodiment, the cartridge is a prefilled cartridge, adapted to be received in the cylindrical shell of the device. According to an embodiment, the cartridge is unidose and the needle assembly contains exactly the amount of second composition to be mixed with the unidose cartridge.

In a preferred embodiment of the invention, the first and the second composition are sterile. According to an embodiment, the needle assembly, including the second composition, is sterile. According to an embodiment, the injection device of the present invention is sterile.

Other characteristics, objects and advantages of the invention will become apparent from the following detailed description provided with reference to the attached drawings, which represent preferred embodiments, by way of non-limiting examples.

### BRIEF DESCRIPTION OF THE DRAWINGS

FIG. 1 is a perspective view of the device of the invention.

FIG. 2A is a side-cross section view of cannula 3, wherein the second composition is in the form of a powder coated inside the cannula, i.e. onto the interior walls of the cannula.

FIG. 2B is a side-cross section view of cannula 3, wherein the second composition is in the form of a powder coated outside the cannula, i.e. onto the outer walls of the cannula.

FIG. 2C is a side-cross section view of cannula 3, wherein the second composition is inside the cannula but not coated on the walls of the cannula.

FIG. 3 is a side-cross section view of a particular embodiment of the invention where the second composition is released within the cartridge or cylindrical barrel.

FIG. 4A is a side-cross section view of an embodiment of the invention, wherein the device comprises a needle assembly defining a hollow chamber.

FIG. 4B is a side-cross section view of an embodiment of the invention, wherein the needle assembly defines a hollow chamber and also comprising two needles.

FIG. 4C is a side-cross section view of an embodiment of the invention, wherein the needle assembly defines a hollow chamber and the connection between the cartridge and the hollow chamber is a corridor.

FIG. 5 is a side-cross section view of a particular embodiment of the invention including a hollow chamber and where the cannula 3 has an orifice located within the hollow chamber.

FIG. 6 is a side-cross section view of a particular embodiment of the invention where the device includes two imbricated needles.

FIG. 7A to 7D are side-cross section views of a particular embodiment of the invention comprising a hub in three parts (lower part, upper part and piston), a seal, and two cannulas.

FIG. 8A to 8D are side-cross section views of a particular embodiment of the invention comprising a hub comprising two parts, and three cannulas.

### DETAILED DESCRIPTION OF THE DRAWINGS

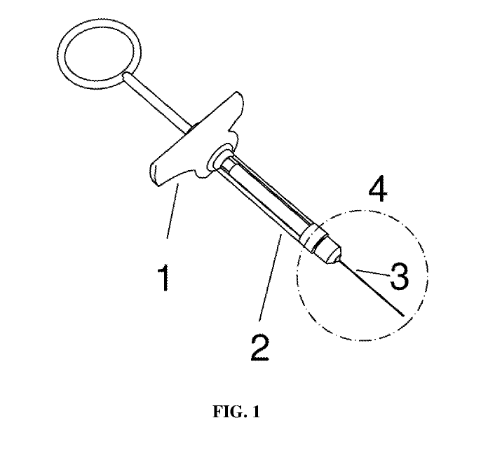

As shown in FIG. 1, the device of the invention may be a syringe 1 comprising a cylindrical barrel or a cartridge 2 including a first composition and a needle assembly 4, including a second composition 6, said needle assembly including at least one cannula 3 and at least one hub 7.

According to one embodiment, the second composition 6 may be a pH-adjuster, and the first composition may be a composition not having a physiological pH. The second composition 6 is such that it immediately dissolves when put into contact with the first composition released from the cartridge or the cylindrical barrel of the syringe.

According to an embodiment of the invention, the second composition 6 may be associated with the cannula 3, which means that it is within cannula 3 and/or on the outer walls of the cannula 3. In this embodiment, when the second composition 6 is on the outer walls of the cannula 3, the coated outer walls are of course located on the part of the cannula that is within the hub and/or that will penetrate into the cartridge or the cylindrical barrel.

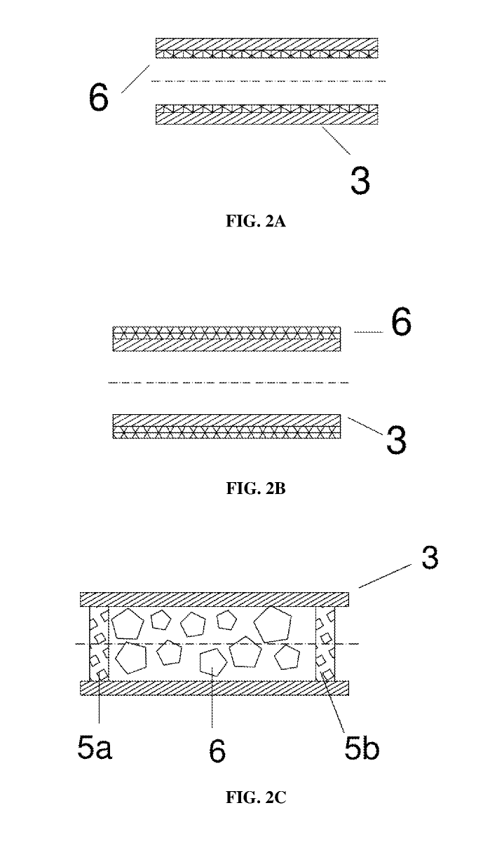

In a first embodiment, the second composition 6 is in a solid form, for example in the form of a powder, coated in the inside of the cannula 3 (see FIG. 2A). The first and the second composition may be contacted and mixed in the cannula, when the operator pushes the plunger and triggers the passage of the first composition through the internally coated cannula.

In a second embodiment, the second composition 6 is in a solid form, for example in the form of a powder, coated in the outside of the cannula 3 (see FIG. 2B). In this embodiment, as mentioned above, the coated part of the cannula is of course located so that the first and second compositions are mixed prior to injection.

In a third embodiment (see FIG. 2C), the second composition 6 is inside the cannula 3 but not coated on the walls of the cannula 3. In this embodiment, the first and the second composition may also be contacted and mixed in the cannula 3, when the operator pushes the plunger and triggers the passage of the first composition through the internally coated cannula 3; in this embodiment, the second composition 6 may be in a liquid or in a solid form.

The device of the invention may comprise at least one filter 5, preferably a back filter 5a or a front filter 5b, or both. The filter 5 may have several functions, among which the prevention of any undesirable movement of the second composition 6 located in the cannula.

In another embodiment (not represented), the second composition 6 is in a solid form and is coated both in the inside of the cannula 3, and in the outside of the cannula 3.

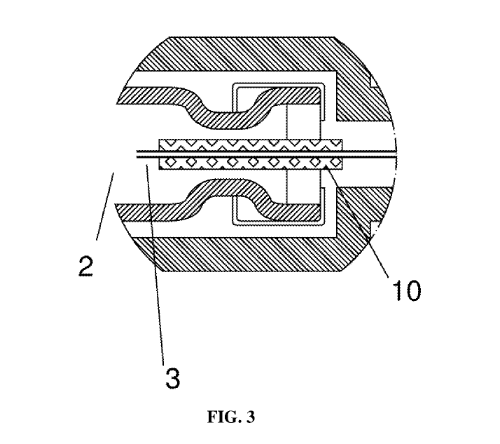

FIG. 3 shows an embodiment where the cannula 3 may be externally coated with the second composition 6 or may have a container 10 surrounding the cannula 3 such that the container 10 contains the second composition 6 and is susceptible to release the second composition 6. In an embodiment, the part of cannula 3 which is externally coated or surrounded by container 10 is the part of the cannula 3 which is adapted to be inserted in the cartridge or cylindrical barrel 2 containing the first composition. In the embodiment, the release of the second composition 6 may be performed by the mere action of inserting cannula 3 in the cartridge or cylindrical barrel 2. In an embodiment container 10 may open or dissolve when contacted with the first composition.

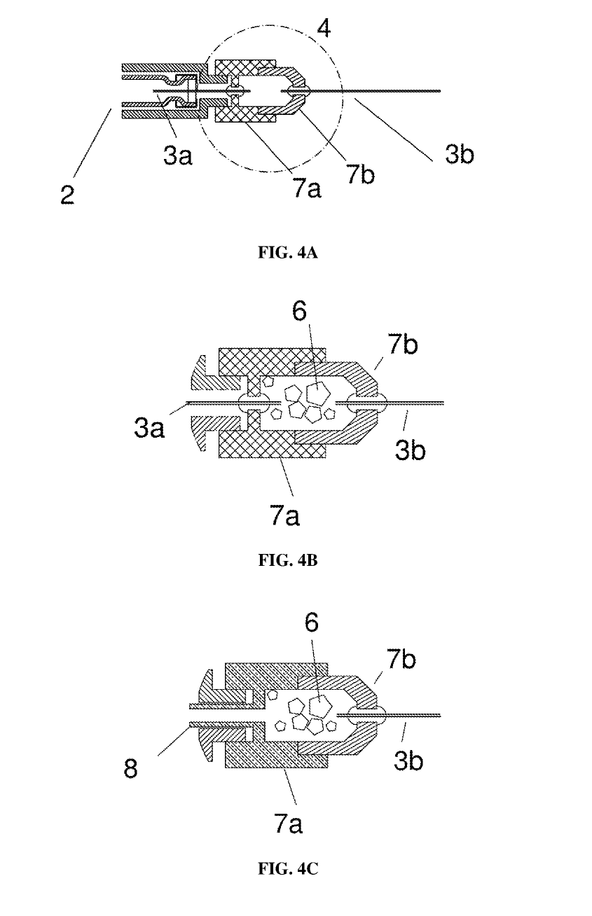

FIGS. 4A, 4B and 4C show a particular embodiment of the invention wherein the device includes a hub in two pieces 7a and 7b, hub piece 7a being connected to hub piece 7b, so that hub piece 7a and hub piece 7b define a closed chamber, filled with the second composition 6. Hub piece 7a connects with the needle support member 9 on one end, and to hub piece 7b on the other end, and hub piece 7b is attached to the cannula 3 on the other end.

FIG. 4A shows an embodiment of the invention, where the device comprises a specific needle assembly 4 defining a hollow chamber where the second composition 6 is placed and where the mixture occurs.

In this embodiment, the first composition is released from the cartridge 2 or cylindrical barrel towards the chamber defined by connected hub piece 7a and hub piece 7b and thus contacted with the second composition 6 in said chamber.

In one embodiment (see FIG. 4A and FIG. 4B) the cannula 3a and the cannula 3b may not be connected. In other words, the device may include two distinct cannulas. Through the first cannula 3a and upon the action of the operator onto the plunger, the first composition is released into the chamber defined by hub pieces 7a and 7b and contacted with the second composition 6. Through second cannula 3b, the mixture is expelled out of the device through a further action of the operator on the plunger.

In another embodiment (see FIG. 4C), the first composition is released in the chamber through a corridor 8.

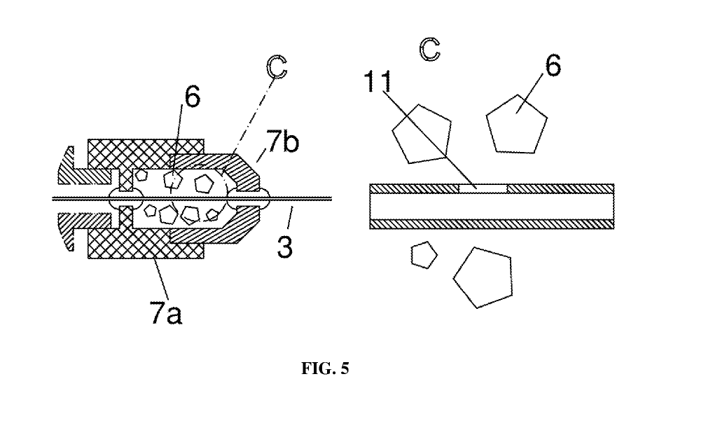

FIG. 5 shows a particular embodiment of the invention wherein the device includes a hollow chamber defined by hub piece 7a and hub piece 7b, as described above in FIG. 4, and where the cannula 3 has an orifice located within the hollow chamber. This orifice is a lateral opening 11 in the cannula, through which first composition and second composition may be put in contact.

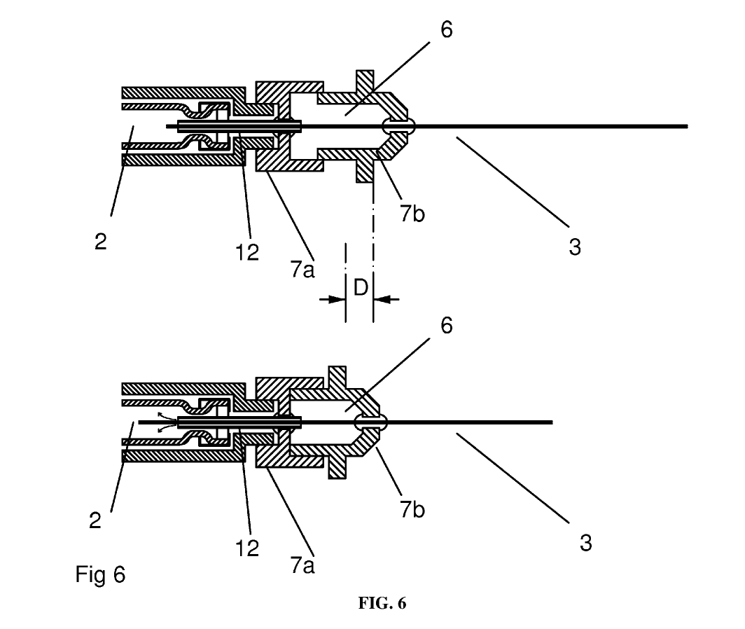

FIG. 6 show a particular embodiment of the invention, wherein the device comprises the cartridge 2 or cylindrical barrel, a first cannula 3, a second cannula 12 surrounding part of cannula 3 and a hub in two pieces 7a and 7b defining a hollow chamber, as already described in the other figures. Cannula 3 passes through cannula 12. The hollow chamber is filled with the second composition 6. In this embodiment, advantageously, the second composition is a liquid. In one embodiment, hub piece 7b may be slidably movable in axial direction towards hub piece 7a. When hub piece 7b is pushed backward to hub piece 7a, the volume of the chamber defined by the two hub pieces 7a and 7b decreases, and second composition 6 is pushed into the cartridge 2 or cylindrical barrel by means of outer needle 12. Excess of liquid may release from cartridge 2 or cylindrical barrel by means of cannula 3. In this embodiment, the content of the hollow chamber, i.e. the second composition 6, is brought into the cartridge or cylindrical barrel 2 containing the first composition.

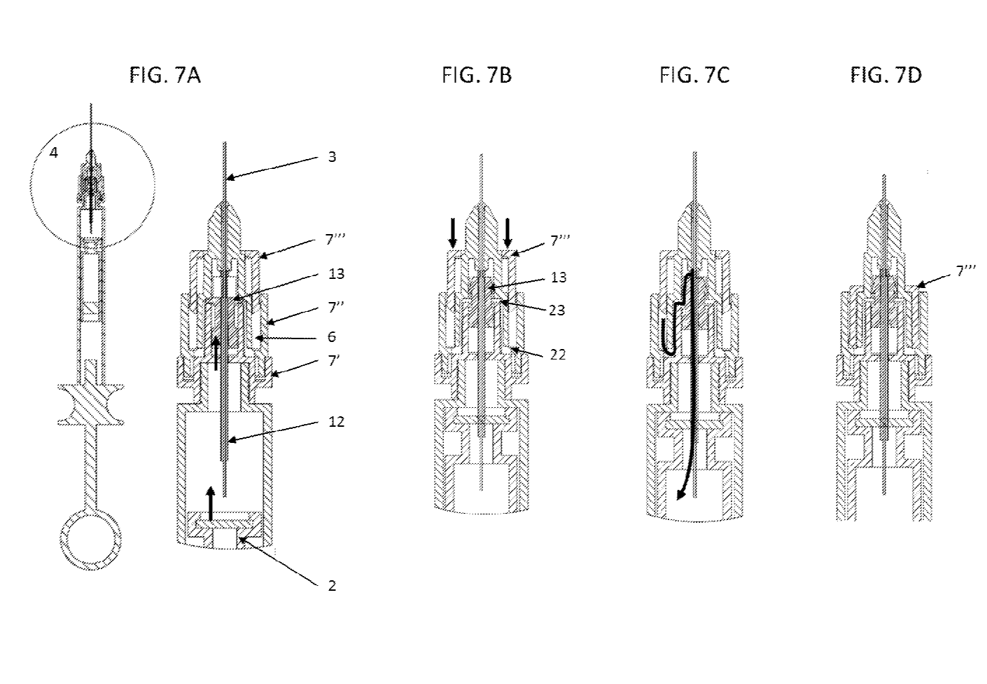

FIGS. 7A, 7B, 7C and 7D show a particular embodiment of the invention, wherein the needle assembly comprises a cannula 3 extending from outside the part of the hub directed to the patient to outside the part of the hub directed to the needle support member; part of said cannula 3 being surrounded by a further cannula 12 extending from inside the hub to outside the part of the hub directed to the needle support member;
and wherein the hub is divided into three pieces, the lower hub piece 7′, the upper hub piece 7″ and the piston 7′″; the lower hub piece 7′ being connectable to a cylindrical shell or a cylindrical barrel; the upper hub piece 7″ being comprising at least one opening 22; the piston 7′ being a piston slidably movable in axial direction towards upper hub piece 7″ and fixed onto said upper hub piece 7″;
the upper hub piece 7″, the lower hub piece 7′ and cannula 12 being connected by a seal 13; the upper hub piece 7″, the seal 13 and the piston 7′″ defining a hollow chamber; the second composition 6 being contained in said hollow chamber.

In this embodiment, advantageously, the second composition 6 is a liquid.

The device according to this embodiment presents the advantage to maintain pressure inside the hollow chamber containing the second composition. This is of particular interest when the second composition is a buffer solution containing bicarbonate as the following equilibrium exists:

2HCO₃⁻═CO₃²⁻+CO₂+H₂O

In an open system, carbon dioxide may therefore escapes, resulting in changes in the pH of the solution. In the case of an initial buffered solution, escape of carbon dioxide renders the solution no more buffered. The device of the present embodiment allows therefore keeping a buffered solution during storage.

The device according to this embodiment also presents the advantage to prevent water evaporation when the second composition is a solution, especially a buffer solution, allowing to preserve initial properties of the composition.

On FIG. 7A is shown the device of this particular embodiment prior to the mixture. The seal 13 is initially tightly compressed by the upper and lower hub pieces 7′ and 7″. When a cartridge 2 is entered onto the needle assembly, the seal of the cartridge diaphragm exercises a force on the surrounding cannula 12 and move both the surrounding cannula 12 and the seal 13 forward. These movements are represented by the two vertical up arrows. Therefore the device of this particular embodiment is only active when a cartridge 2 is in place.

According to an embodiment, seal 13 is impermeable to gas, especially to carbon dioxide.

On FIG. 7B is shown the device of this particular embodiment after the adaptation of the cartridge 2 on the needle assembly. As explained above, when the cartridge is adapted on the present device, the seal 13 moves in a forward position as represented on FIG. 7B. This movement liberates openings 23 between the seal 13 and the upper hub piece 7″. In one embodiment, ribs are presents inside the upper hub piece 7″ enabling the seal 13 to fold. The displacement of the seal 13 allows the second composition 6 to circulate through the device into the cartridge 2. The piston 7′″ may be moved toward the hub piece 7″ so that the circulation of second composition 6 being facilitated by pressure elevation in the hallow chamber. The movement of the piston 7′″ is represented by the two vertical dawn arrows.

On FIG. 7C is shown the circulation of the second composition 6 inside the device of this particular embodiment when piston 7′″ is moved. The circulation of the second composition is represented by the arrow. When piston 7′″ is pushed backward to upper hub piece 7″, the volume of the chamber defined by the two hub pieces decreases. The second composition 6 is pushed from the hollow chamber through openings 22 and 23 and moves into the cartridge 2 by means of outer needle 12. Excess of liquid may release from cartridge 2 or cylindrical barrel by means of cannula 3. In this embodiment, the content of the hollow chamber, i.e. the second composition 6, is brought into the cartridge 2 or cylindrical barrel containing the first composition.

On FIG. 7D is shown the device of this particular embodiment when piston 7′″ has been completely moved backward to hub piece 7″.

In this embodiment, the piston 7′″ may be moved either directly with fingers or by moving a protective sheath. For example, the exterior part of the cannula 3 may be protected by a protective sheath (not represented). This protective sheath may be connected to the piston 7′″. In this case, when moving the protective sheath to prepare the device to injection by exposing the needle, the piston 7′″ is moved and the second composition 6 is sent in the cartridge where the two compositions are mixed. The resulting buffered mixture may then be injected to the patient.

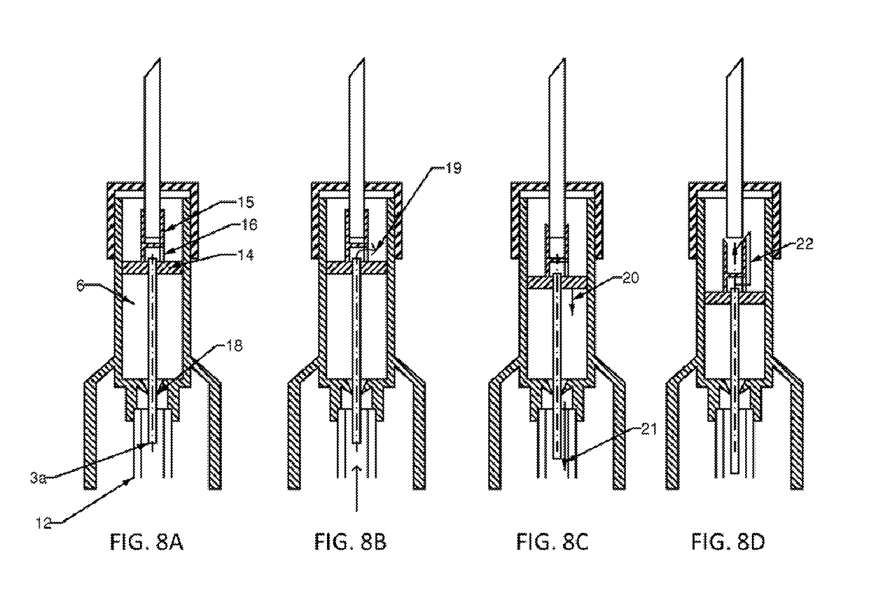

FIGS. 8A, 8B, 8C and 8D show a particular embodiment of the invention, where the hub is separated in two parts by a movable wall 14. On FIG. 8A is shown the device of the invention prior to the mixture; the device comprises an internal cannula 3a and a surrounding cannula 12 extending from the hub to the cartridge, and an outer cannula extending outward for use for injection to the patient; the hub is separated in two parts by a movable wall 14 having a perpendicular protusion 15. The protrusion 15 seals the inner end of outer cannula. In the walls of protrusion 15 is located an orifice 16. The outer second composition 6 is encapsulated, preferably in a sterile way, within the lower part of the hub, the upper part of the hub being empty. The encapsulation of second composition was made possible thanks to the presence of a lid 18 located between the lower part of the hub and the cannula 12. The lid 18 is also capable, when a pressure is applied onto said lid 18, to have the second composition pass through to cannula 12.

On 8B, is shown that, when the plunger is pushed, the first composition is brought to the upper part of the hub through cannula 3a and through hole 16. Reference 19 points to an arrow intended to show the movement of the first composition. When the upper part is being filled, the pressure of the first composition pushes backwards the movable wall 14 and (1) liberates the end of the outer cannula from its seal and (2) restricts the volume of the lower part of the hub.

As shown in FIG. 8C, the restriction of the lower part of the hub results in applying a pressure on lip 18, which results in second composition 6 passing through to cannula 12 and then to the cartridge, where second composition 6 mixes with first composition. In FIG. 8C, reference 20 points to a backward arrow intended to show the movement of wall 14. Reference 21 points to a backward arrow intended to show the movement of second composition 6.

As shown in FIG. 8D, continuous push on the plunger will then result to reinjecting the mixture in the upper part of the hub through cannula 3a and hole 16, from where it can be injected through outer cannula to the patient.

1.-13. (canceled)

14. An injection device comprising:
a generally cylindrical shell in which a cartridge may be lodged or a generally cylindrical barrel;
at one first end of the generally cylindrical barrel or the generally cylindrical shell, a plunger, the plunger being mounted on the barrel or shell; and
at a second end opposite the first end of the generally cylindrical barrel or the generally cylindrical shell, is provided a needle assembly support member that is adapted to receive a needle assembly.

15. The injection device of claim 14, further comprising a needle assembly mounted on the needle assembly support member.

16. The injection device of claim 15, wherein the needle assembly is further defined as comprising:
at least one hollow cannula for transportation of liquid from a barrel or cartridge to the body of a patient; and
at least one hub surrounding part of the cannula and attached to the cannula.

17. The injection device of claim 16, further defined as comprising at least two cannulas.

18. The injection device of claim 16, wherein the hub comprises a hollow part.

19. The injection device of claim 18, wherein an injectable composition is comprised in the needle assembly.

20. The injection device of claim 15, further defined as comprising a first injectable composition, which is within the cylindrical barrel or within the cartridge lodged in the cylindrical shell and a second injectable composition, which is associated with the needle assembly, and is adapted to mix the first and the second composition, such that the mixture of said first and said second composition occurs in the hub, in the cartridge or barrel, or in the cannula.

21. The injection device of claim 20, wherein:
a cannula extends from outside a part of the hub directed to a patient to outside a part of the hub directed to the needle support member, part of said cannula is surrounded by a further cannula extending from inside the hub to outside the part of the hub directed to the needle support member;
the hub is divided into three pieces, a lower hub piece, an upper hub piece and a piston, the lower hub piece is connectable to a cylindrical shell or a cylindrical barrel, the upper hub piece comprising at least one opening, the piston is a piston slidably movable in axial direction towards upper hub piece and fixed onto said upper hub piece; and
the upper hub piece, the lower hub piece, and the cannula are connected by a seal, the upper hub piece, the seal and the piston define a hollow chamber, and the second composition is contained in said hollow chamber.

22. The injection device of claim 20, wherein the first composition is an injectable composition having a non-physiological pH and the second composition is a buffer of the first composition.

23. The injection device of claim 22, wherein the buffer comprises NaHC0₃, a salt of citric acid, and/or a salt of phosphoric acid.

24. The injection device of claim 22, wherein the first composition is a dental anesthetic

25. The injection device of claim 24, wherein the dental anesthetic comprises lidocaine, mepivacaine, prilocaine, and/or articaine.

26. The injection device of claim 22, wherein the second composition has a pK of 6.5 to 7.8.

27. The injection device of claim 26, wherein the second composition has a pK of 7 to 7.4.

28. The injection device of claim 20, wherein the second composition is in the form of a solid or a liquid.

29. The injection device of claim 28, wherein the second composition is a powder, a solution, or an emulsion.

30. The injection device of claim 20, wherein the second composition is comprised in the needle assembly.

31. The injection device of claim 30, wherein the second composition is disposed on or in the cannula or hub.

32. A needle assembly comprising:
at least one hollow cannula for transportation of liquid from a barrel or cartridge to the body of a patient; and
at least one hub surrounding part of the cannula and attached to the cannula.

33. The needle assembly of claim 32, further defined as comprising at least two cannulas.

34. The needle assembly of claim 32, wherein the hub comprises a hollow part.

35. The needle assembly of claim 34, wherein an injectable composition is comprised in the needle assembly.

36. A method for mixing a first injectable liquid pharmaceutical composition and a second injectable composition within an injection device comprising:
obtaining an injection device of claim 14; and
mixing a first injectable liquid pharmaceutical composition and a second injectable composition in the injection device.

37. The method of claim 36, wherein the mixing occurs in the cartridge or barrel, in the cannula, or in the hub.

38. The method of claim 37, further comprising injecting the mixed first and second compositions into a patient.
